(19) United States
(12) Patent Application Publication
(10) Pub. No.: US 2012/0220938 A1
(43) Pub. Date: Aug. 30, 2012

(54) INTRAVENOUS FLUID HEATER

(75) Inventors: Darrel Burton Saunders, Windsor (CA); Robert Charles Echlin, Windsor, Ontario (CA)

(73) Assignee: NORTH AMERICAN RESCUE, LLC, Greer, SC (US)

(21) Appl. No.: 13/505,617

(22) PCT Filed: Jun. 9, 2011
(86) PCT No.: PCT/US2011/039741

§ 371 Date: May 2, 2012

Related U.S. Application Data
(60) Provisional application No. 61/353,469, filed on Jun. 10, 2010.

(51) Int. Cl. A61M 5/44

(52) U.S. Cl. 604/114; 604/113

(57) ABSTRACT

A compact and highly efficient intravenous fluid heater includes a heat exchanger assembly, sensors for sensing respective temperatures of entering and exiting fluids of the flow path, a controller for controlling the heat generated by the heat exchanger assembly, based upon the temperatures of the exiting fluids, heating of the fluid in the flow path by the heating element so as to cause the fluid in the flow path to be substantially uniformly heated to a desired infusion temperature prior to exiting the heater. The heat exchanger assembly uses thin film heating elements having a predetermined set temperature and that transfers heat directly to a standard intravenous tube coil wrapped around the battery housing. The intravenous fluid heater is located in a housing with the intravenous fluid lines extending there from for standard connection with an intravenous line or source of intravenous fluid.

### CROSS REFERENCE TO RELATED APPLICATIONS

This application claims the benefit of priority from U.S. Provisional Application No. 61/353,469.

### BACKGROUND OF THE INVENTION

1) Field of the Invention

The present invention relates to an intravenous fluid warmer and, in particular, relates to an intravenous fluid warmer that is particularly suitable for use in warming fluids and the management of hypothermia in wilderness, combat and other extreme weather and/or remote environments and locations.

2) Description of Related Art

Intravenous (IV) fluid heaters or warmers have traditionally been powered by an AC power source because of the high power required to heat IV fluids. Battery powered IV fluid warmer have heretofore had poor performance because of the battery sources which have been available.

One known battery-powered blood or IV fluid warmer is marketed as the Thermal Angel® and is produced by Estill Medical Technologies, Inc. having an address of 4144 N. Central Expressway. Suite 260 Dallas, Tex. 75204 and was found on the website http://www.thermalangel.com. The TA-200 model Thermal Angel® device is disclosed as using a 12 volt, rechargeable, lead acid battery weighing 6.25 pounds. This battery requires an external charger and thus requires an extra piece of equipment for operational use. The TA-200 model Thermal Angel® battery has a heating capacity of about 2 to 4 liters of room temperature IV fluid or about 2 to 3 liters of blood. It is understood at this time that the TA-200 model Thermal Angel® battery cannot be charged while the fluid warmer is in use. Further, it is understood that the TA-200 model Thermal Angel® battery has a complete charge time of approximately twelve hours and cannot be fast charged. In addition, The TA-200 model Thermal Angel® battery has only a simplified gauge which is only accurate when the battery is not in use.

Another known battery-powered, IV fluid warmer relates to a lithium polymer (LiPo) battery because LiPo batteries were known to have extremely low internal impedance and to be particularly suitable for high current applications. For example, US Patent Application No. 2007/0105010 Al, to Cassidy, published May 10, 2007 and entitled Lithium Polymer Battery Powered Intravenous Fluid Warmer, discloses such a known LiPo as having a relatively high energy density, no exhibited memory effects, and to be environmentally safe. In particular, the US 2007/0105010 reference also discloses a battery management system for such an IV heater which addresses potential issues with LiPo batteries because they may become dangerous if overcharged or over-discharged and in such circumstances the batteries can explode or catch fire.

Other prior art techniques and devices exist for warming fluids to be infused intravenously into a body, such as a human or other animal. One such conventional device is disclosed in U.S. Pat. No. 5,245,693 (“the '693 patent”). The '693 patent is directed to an intravenous fluid heater that includes a disposable cassette containing a heat exchanger. The preferred embodiment of the heat exchanger disclosed in the '693 patent includes a passageway-defining inner layer sandwiched between a pair of flexible, metal foil membranes. The inner layer defines an extended, e.g., serpentine, path for fluid to be warmed, and serves to space apart and insulate the metal foil membranes from one another. Inlet and outlet ports to the serpentine fluid path are defined in one of the two foil membranes. Heat generated by heating elements which sandwich the heat exchanger is transferred through the metal foil membranes to the fluid flowing through the serpentine path. The heating elements are designed to be graduated, that is, to generate more heat in the area of the inlet portion of the serpentine path than in the area of its outlet.

A yet further disadvantage of the heating arrangement disclosed in the '693 patent is that although means are included for reducing gas bubble formation in the infusion fluid, no means are provided for automatically determining whether such bubbles are present in the fluid or whether there has been reduction in fluid flow rate, and for taking appropriate action (e.g., providing warning and/or stopping fluid flow into the patient) in the event such conditions are determined to be present. As will be appreciated by those skilled in the art, if left unchecked these types of conditions can be, at minimum, deleterious to patient well-being, and at most, life-threatening.

Another conventional infusion fluid warming device is disclosed in U.S. Pat. No. 5,254,094 (“the '094 patent”). In the arrangement disclosed in the '094 patent, a box which may be attached to a patient's arm is provided. Two chambers are included in the box, containing a heat exchanger element constructed from a continuous length of stainless steel tubing in the form of two parallel coils which are connected to each other by a straight length of tubing. The box includes a passage between the chambers such that a warming fluid may be introduced through an aperture in the box into one of the chambers, flow into the other chamber, and then exit the warmer through another aperture in the box. The infusion fluid to be warmed is supplied to the coils through a first flexible plastic inlet tube and discharged for infusion into a patient through a second flexible plastic tube. The warming fluid is supplied via fluid supply tubing to the box from a separate fluid source that is not dimensioned or suitable for being worn by the patient, such as a water heater. A temperature sensor located in the infusion fluid path between the box and the infusion sites may be provided for generating signals indicative of the temperature of the infusion fluid for provision to a microprocessor contained in the same unit comprising the water heater. The microprocessor also receives outputs from a water temperature sensor and controls the water heater, based upon the outputs from these sensors and a desired infusion fluid temperature set by the user, so as to maintain the heating water at a temperature for heating the infusion fluid to the desired temperature.

Disadvantageously, use of a warming fluid/infusion fluid type of heat exchanger, and a warming fluid heater that is remote from the heat exchanger and not wearable by the patient, make '094 patent's arrangement bulky, and relatively difficult to move and set up for use. Also disadvantageously, if even a single crack, pin-hole, imperfect seal, or other opening exists in the infusion fluid tubing/fittings in the heat exchanger, the infusion fluid may become contaminated with the warming fluid. Other examples of infusion fluid warming prior art are disclosed in U.S. Pat. Nos. 5,381,510, 4,731,072, 3,443,060, 3,475,590, 3,485,245, 3,590,215, 3,614,385, 3,640,283, 3,853,479, 4,038,519, 4,108,146, 4,167,663, 4,293,762, 4,309,592, 4,938,279, 4,847,470, 4,574,876, 3,399,536, 4,962,761, 5,125,069, 4,908,014, 4,906,816, 4,844,074, 4,707,587, 4,759,749, 4,782,212, 4,801,777, 4,680,445, 4,678,460, 4,532,414, 4,464,563, 4,314,143, 4,356,383, and, 4,878,537. While these and other known devices and technologies offer various advantages and benefits, they also suffer from the aforesaid and/or other disadvantages and drawbacks.

Accordingly, there remains a significant and long felt need to have an intravenous fluid warmer that will provide greater benefits and advantages in the management of hypothermia in wilderness, combat and other extreme and/or remote environments and locations.

### SUMMARY OF THE INVENTION

In one exemplary embodiment, there is disclosed a heat transfer apparatus for use with an intravenous fluid to be administered to a body. In one aspect, the heat transfer apparatus includes a housing configured for having an intravenous line to enter at a first point and to exit at a second point; a heat transfer element for producing heat to be transferred with the intravenous line to change the temperature of the intravenous fluid flowing through the intravenous line; and a power source for supplying energy to the heat transfer apparatus, the power source being coupled with the heat transfer apparatus and being located at least partially within the housing.

In one alternate exemplary embodiment, there is disclosed an apparatus for changing the temperature of a fluid to be administered intravenously to a body using an intravenous line, the apparatus includes a housing configured for having the intravenous line enter and exit the housing, a heat transfer element for transferring heat with the intravenous line to change the temperature of the fluid in the intravenous line, and a power source for supplying energy to the heating element to produce heat, the power source being located within the housing. In another alternate exemplary embodiment, there is disclosed a portable apparatus for providing energy to heat an intravenous fluid, the portable apparatus including a housing having an opening for receiving a length of a flexible intravenous tube, a heat transfer element for producing heat to be transferred with the intravenous line to change the temperature of the intravenous fluid flowing through the intravenous line, and an energy source comprising a battery cell arranged to provide electrical power to the heat transfer element and the intravenous tube is routed for a given length concentrically around the one or more battery cells.

In one alternate exemplary embodiment, there is disclosed a portable apparatus for providing energy to heat an intravenous fluid, the portable apparatus includes a housing having an opening to receive a length of a flexible intravenous tube for connecting a source of intravenous fluid to a body, an energy source comprising one or more battery cells, a heating element for converting energy from the battery cells to heat for heating the intravenous fluid flowing in the intravenous tube, and wherein the intravenous tube is at least partially wrapped around the one or more battery cells. In one exemplary embodiment, the intravenous tube is comprised of a metal material and, in particular, includes aluminum. In one exemplary embodiment, the intravenous tube comprises a stainless steel material.

In one alternate exemplary embodiment, there is disclosed portable apparatus for providing energy to heat an intravenous fluid including a housing having an opening to receive a length of a flexible intravenous tube for connecting a source of intravenous fluid to an intravenous catheter, a heat transfer element for transferring heat with the intravenous tube and the intravenous fluid flowing in the intravenous tube and an energy source for supplying energy to the heat transfer element; and an energy source receptacle having at least a portion of the energy source located therein, the energy source receptacle being located within the housing and the intravenous tube has at least a portion extending between the heat transfer element and the energy source receptacle. In one particular alternate exemplary embodiment, the intravenous tube is coil wrapped for a given length concentrically around the energy source receptacle containing the one or more battery cells.

In one alternate exemplary embodiment, there is disclosed a portable intravenous tube system including a heating apparatus for heating an intravenous fluid including an apparatus for providing energy for use in transferring heat with the intravenous fluid, a housing for receiving at least a portion of the intravenous tube, an energy source for transferring heat to the intravenous fluid flowing in the intravenous tube; and wherein the intravenous tube is coiled for a given length within the housing to increase the total heat transferred to the intravenous fluid flowing through the intravenous tube.

In one particular exemplary embodiment, there is disclosed an intravenous thermal device that includes a thin-film heater element consisting of a relatively thin polyamide, PTC or polyester film impregnated with a resistive carbon layer onto which conductive busses are printed. The film of the heater element itself is less than 0.5 mm thick with unparalleled strength, flexibility and reliability. Critically, this heater technology draws far less power than competitive alternatives allowing for extended battery life and lightweight, highly portable designs. In one exemplary embodiment, the heater material of the heater elements may be directly applied to the housing members using a spray and/or deposition technique. In one alternate embodiment, it is possible to use a single layer carbon-based resistive film that is directly applied to the outer surface of the inner housing.

In one alternate exemplary embodiment, the intravenous fluid warming device has particular additional practical uses due to its improved portability, power efficiency and flexibility supporting improved patient outcomes. Further, in one alternate embodiment, the intravenous fluid warming device is capable of running off of disposable or rechargeable batteries while providing appropriate and necessary performance specifications for use in relatively extreme climate conditions.

In one exemplary embodiment, a heat transfer apparatus is particularly useful for heating an intravenous fluid to be administered to a body, the heat transfer apparatus includes a housing configured for having an intravenous line enter at a first point and exit at a second point, an energy source for supplying energy to the heat transfer apparatus to generate heat, wherein the energy source is located within the housing, and a heat transfer element for producing heat to be transferred with the intravenous line to change the temperature of the intravenous fluid flowing through the intravenous line. In one alternate exemplary embodiment, the heat transfer apparatus is located proximal the housing and the energy source is centrally located within housing and intravenous line carrying the intravenous fluid is located between the heat transfer apparatus and the energy source. Further, the heat transfer element and the intravenous line are pressed together to increase the effective heat transfer with the fluid in the intravenous line in one embodiment. In one alternate embodiment, the heat transfer device further includes an inner housing member located within the housing and having the energy source located within the inner housing and a heat transfer element on the exterior of the inner housing and one the interior of the outer housing and the intravenous line contacts the heating elements on the inner and outer housings.

In one exemplary embodiment, the exterior side of the inner housing has a substantially round cross section and is located concentrically with the interior side of the exterior housing to define a space there between and the heat transfer elements and the intravenous line are located within the space. In one particular embodiment, the space is substantially cylindrically shaped and the intravenous line is coil wrapped within the space. In one particular embodiment, the intravenous line is coil wrapped around the inner housing and each wrap is spaced a predetermined amount to increase the heat transfer to an intravenous fluid within the intravenous fluid line.

In one exemplary embodiment, the heat transfer apparatus includes at least one temperature sensor, and preferably two, for monitoring the temperature of the intravenous fluid exiting (and entering) the heat transfer apparatus and the heat transfer apparatus includes a display for indicating the status of the temperature sensor—that is representative of the temperature of the intravenous fluid at that location. The heat transfer apparatus is responsive to the temperature sensor to control the energy from the energy source being supplied the heat transfer elements for maintaining the temperature of the fluid within a defined temperature range. In one exemplary embodiment, the heat transfer apparatus includes a controller that is also responsive to a flow sensor for measuring the flow of the intravenous fluid within the apparatus and a pressure sensor for measuring the pressure within the apparatus. The controller receives inputs from the pressure and flow sensors for determining the control of the heat transfer apparatus while managing the temperature of the intravenous fluid exiting the heat transfer apparatus. In one exemplary embodiment, the housing includes a portion that is at least partially translucent and the display is arranged in the housing to be viewable through the translucent portion of the housing. Further, in one exemplary embodiment, the display also indicates the status of the energy source and provides a display of the temperature of the intravenous fluid.

An apparatus for changing the temperature of a fluid to be flowing in an intravenous line to be administered intravenously to a body, the apparatus comprising: a housing configured for having the intravenous line enter and exit the housing; a heat transfer element for transferring heat with the intravenous line to change the temperature of the fluid in the intravenous line; an energy source for supplying energy to the heating element to produce heat, the energy source being located within the housing; a temperature sensor for monitoring the temperature of the fluid exiting the apparatus; a controller coupled to the housing for controlling the supply of energy from the energy source to the heating element in response to the temperature sensor; and a display coupled to the apparatus, the display for indicating the status of the apparatus.

In one exemplary embodiment, the heat transfer apparatus also includes a vent system located within the housing, the vent system is coupled within the intravenous line to vent gas from the intravenous fluid prior to the intravenous fluid exiting the heat transfer apparatus. In one exemplary embodiment, the vent system vents gas from the intravenous fluid to balance the pressure within the apparatus with the ambient pressure outside of the apparatus.

In one exemplary embodiment, there is disclosed a portable apparatus for providing energy to heat an intravenous fluid having a housing having an opening to receive a length of a flexible intravenous tube for connecting a source of intravenous fluid to a body, an energy source comprising a battery cell, a heating element for converting energy from the energy source to heat for heating the intravenous fluid flowing in the intravenous tube, and wherein the intravenous tube is at least partially wrapped around the battery cell.

In one exemplary embodiment, there is disclosed an intravenous tube heater system for use in austere weather environments wherein the intravenous tube heater includes a heating apparatus for heating an intravenous fluid flowing in the intravenous tube, the heating apparatus including a heating element for transferring heat to the intravenous fluid, a housing coupled to the intravenous tube, an energy source for supplying energy to the heating element, and wherein the intravenous tube heater system is designed to be disposable after a single use. In one exemplary embodiment the intravenous tube heater system wherein the heater system includes an inner housing for containing the energy source and the inner housing, energy source and intravenous tube is a removable unit from the heater system housing.

In one exemplary embodiment, there is disclosed a heated intravenous tube system for use in austere weather environments, the intravenous tube system includes a heating apparatus for heating an intravenous fluid, the heating apparatus including a heating element for transferring heat to the intravenous fluid, a housing coupled to the intravenous tube, an energy source for supplying energy to the heating element, and wherein the intravenous tube has a first end having a first coupling for coupling heated intravenous tube system to one of another intravenous tube and an intravenous bag containing a fluid and a second end having a second coupling for coupling the heated intravenous tube system to one of another intravenous tube and a catheter.

In one exemplary embodiment an intravenous tube heater for use in austere weather environments includes an intravenous tube system, a heating apparatus for heating an intravenous fluid, the heating apparatus including a heating element for transferring heat to the intravenous fluid, a housing coupled to the intravenous tube, an energy source for supplying energy to the heating element, and wherein the intravenous tube heater includes a intravenous tube portion exiting the intravenous tube heater for connecting the intravenous line to a patient, wherein the intravenous tube portion exiting the intravenous tube heater is insulated to limit and/or prevent heat from exiting the intravenous fluid therein.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows an exposed side view of a intravenous fluid heater device with a top half of the housing being translucent to show the inner details of the intravenous fluid heater device according to one exemplary embodiment;

FIG. 2 shows a top view of the intravenous fluid heater of FIG. 1 with the top half of the housing being translucent to show the inner details of the intravenous fluid heater device according to according to one exemplary embodiment;

FIG. 3 shows a partial cut-away side view of an intravenous fluid heater device of FIG. 1 showing the inner details of the intravenous fluid heater device of FIG. 1 according to one alternate exemplary embodiment;

FIG. 4 shows an end view of the intravenous fluid heater device of FIG. 3 according to one alternate exemplary embodiment;

FIG. 5 shows a detailed partial perspective view of a sensor plug connector on the intravenous fluid heater device of FIG. 3 according to one alternate exemplary embodiment;

FIG. 6 shows a top view of the intravenous fluid heater device of FIG. 3 with the housing in an open position showing the inner details of the intravenous fluid heater device according to one alternate exemplary embodiment;

FIG. 7 shows a perspective view of the intravenous fluid heater device of FIG. 6 according to one alternate exemplary embodiment;

FIG. 8 shows an alternate perspective view of the intravenous fluid heater device of FIG. 6 according to one alternate exemplary embodiment;

FIG. 9 shows a partial sectional view of the intravenous fluid heater device taken from Detail B section of FIG. 8 detailing the coupling of the housing portions and the inner energy source receptacle according to one alternate exemplary embodiment;

FIG. 10 shows a perspective view of an intravenous fluid heater device according to one alternate exemplary embodiment;

FIG. 11 shows an exploded perspective view of the intravenous fluid heater device of FIG. 10 according to one alternate exemplary embodiment;

FIG. 12 shows a plurality of views of a bottom housing portion of the intravenous fluid heater device of FIG. 11 according to one alternate exemplary embodiment;

FIG. 13 shows a plurality of views of a top housing portion of the intravenous fluid heater device of FIG. 11 according to one alternate exemplary embodiment;

FIG. 14 shows a plurality of views of an inner housing portion of the intravenous fluid heater device of FIG. 11 according to one alternate exemplary embodiment;

FIG. 15 shows a plurality of views of a temperature sensor of the intravenous fluid heater device of FIG. 11 according to one alternate exemplary embodiment;

FIG. 16 shows a plurality of views of an intravenous line of the intravenous fluid heater device of FIG. 11 according to one alternate exemplary embodiment;

FIG. 17 shows a plurality of views of a tube extension member of the intravenous fluid heater device of FIG. 11 according to one alternate exemplary embodiment;

FIG. 18 shows a plurality of views of a port access member of the intravenous fluid heater device of FIG. 11 according to one alternate exemplary embodiment;

FIG. 19 shows a block diagram of the intravenous fluid heater device of according to one alternate exemplary embodiment;

FIG. 20 shows a perspective view of an intravenous fluid heater device according to one alternate exemplary embodiment;

FIG. 21 shows a side cross-section view of the intravenous fluid heater of FIG. 20 with the showing the inner details of the intravenous fluid heater device according to the exemplary embodiment of FIG. 20;

FIG. 22 shows a partial perspective view of the internal parts of the intravenous fluid heater device of FIG. 20 according to an alternate exemplary embodiment;

FIG. 23 shows an end view of the intravenous fluid heater device of FIG. 20 according to an alternate exemplary embodiment;

FIG. 24 shows a perspective view of the upper housing of the intravenous fluid heater device of FIG. 20;

FIG. 25 shows an end view of the upper housing of FIG. 24;

FIG. 26 shows a perspective view of the lower housing of the intravenous fluid heater device of FIG. 20;

FIG. 27 shows an end view of the lower housing of FIG. 25;

FIG. 28 shows a perspective view of the battery housing of the intravenous fluid heater device of FIG. 20;

FIG. 29 shows a side view of the battery housing of FIG. 28; and,

FIG. 30 is an exploded side view of the intravenous fluid heater device of FIG. 20.

It will be understood by those skilled in the art that one or more aspects of this invention can meet certain objectives, while one or more other aspects can meet certain other objectives. These and other objects and features of the invention will become more fully apparent when the following detailed description is read in conjunction with the accompanying figures and examples. However, it is to be understood that both the foregoing summary of the invention and the following detailed description are of preferred embodiments and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention.

### DETAILED DESCRIPTION OF A PREFERRED EMBODIMENT

With reference to the drawings, the invention will now be described in more detail. An intravenous fluid heater device is dimensioned to be wearable on the patient such as to be located adjacent to an infusion situs (i.e., the location on the patient's body were the fluid is to be infused into the patient), and is also configured to determine automatically when, and to provide a visual warning if, the fluid flow rate in the intravenous fluid heater device falls below a desired minimum threshold therefore, and/or gas (e.g., air) is present in the fluid. The intravenous fluid heater device has particular utility for supplying an intravenous fluid to patient at flow rates of between about 50 ml/hour to about 4500 ml/hour for an intravenous fluid to be heated to an infusion temperature of between about 38 and about 42 degrees Centigrade (° C.) from an input temperature of between about 1° C. and about 42° C. It should be understood that the intravenous fluid heater device of the present disclosure is operational for the infusion of intravenous fluids at other temperatures and flow rates.

The intravenous fluid heater device or assembly of the present disclosure is useful as an intravenous fluid warmer for use in remote, extreme (particularly cold) weather conditions such as in military and civilian settings, such as a battlefield or civilian medical facility remotely located such as in a mountainous or other region. The heater assembly of the present invention is relatively compact and includes internal batteries useful for supplying energy to the intravenous fluid warmer without the need for an external energy source. The heater device or assembly includes a controllable heat exchanger having heating elements and input and output temperature sensors for sensing the temperature of the intravenous fluid entering and existing the intravenous fluid heater device. A controller is used for controlling the supply of electrical energy to the heater elements which controls the amount of heat energy supplied to the intravenous fluid flowing through the heater device in the flow path by heat conduction thereto through the heat exchanger and an intravenous tube wrapped around a battery housing within the housing of the heater device. The intravenous fluid in the intravenous tube flow path is substantially uniformly heated to the desired infusion temperature prior to exiting the heater device.

The controller may be programmed to determine, based at least in part upon one or both of the entering/exiting fluid temperatures, whether the flow rate of fluid is below a desired threshold value therefore, and/or whether gas is present in the flow path. The controller may also be programmed to initiate a warning/indication to a user of the device, and/or to initiate other appropriate action (e.g., closing a valve in the device to prevent further flow of fluid to the patient), when one or both of the aforesaid conditions are present. The at least one heating element of the heat exchanger may include one, two, three or more heating elements as may be appropriate or necessary, and the heat exchanger may also include a plurality of insulating layers.

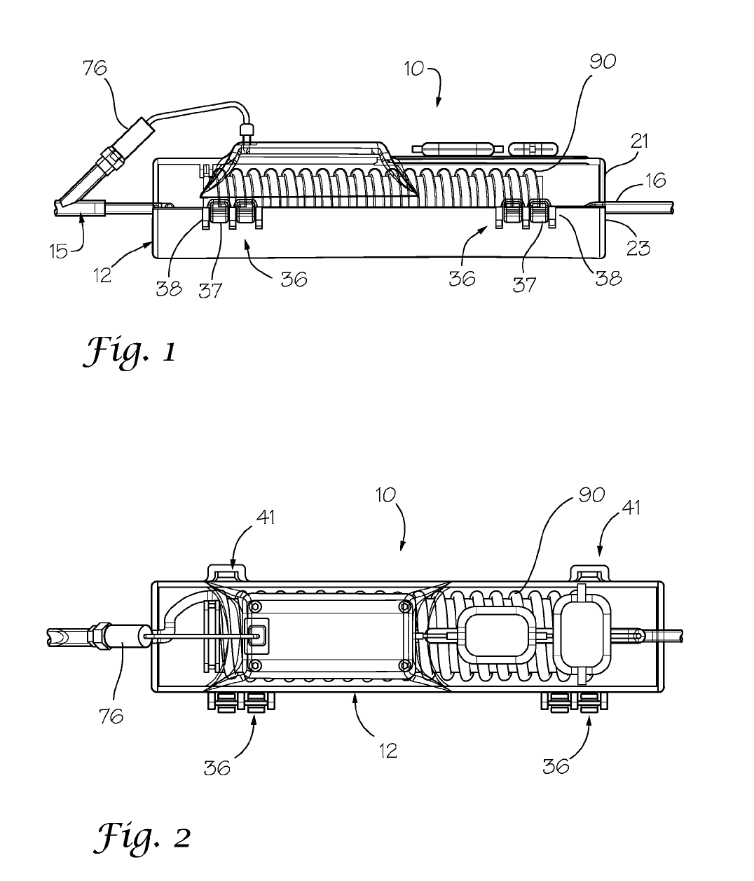

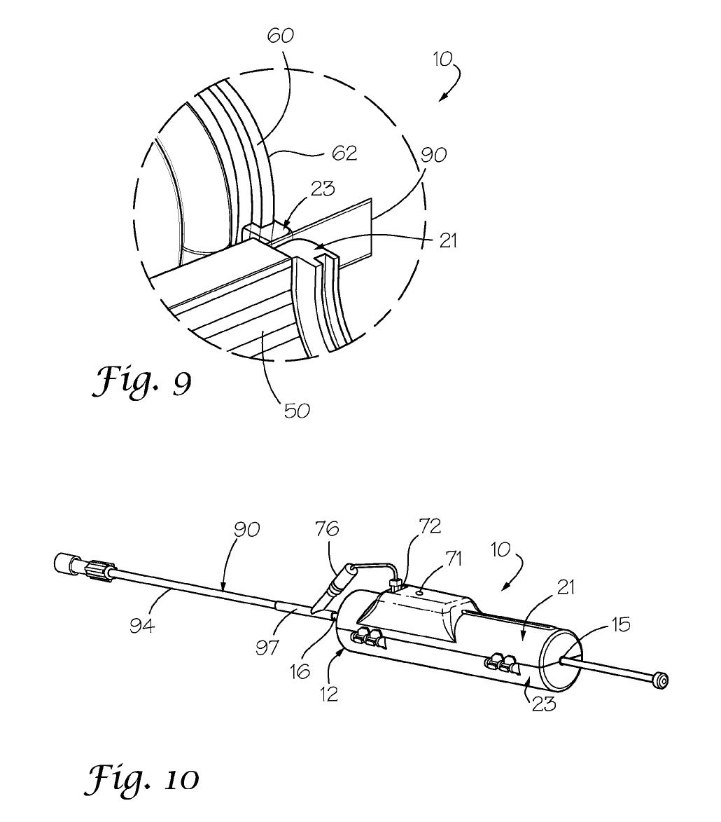

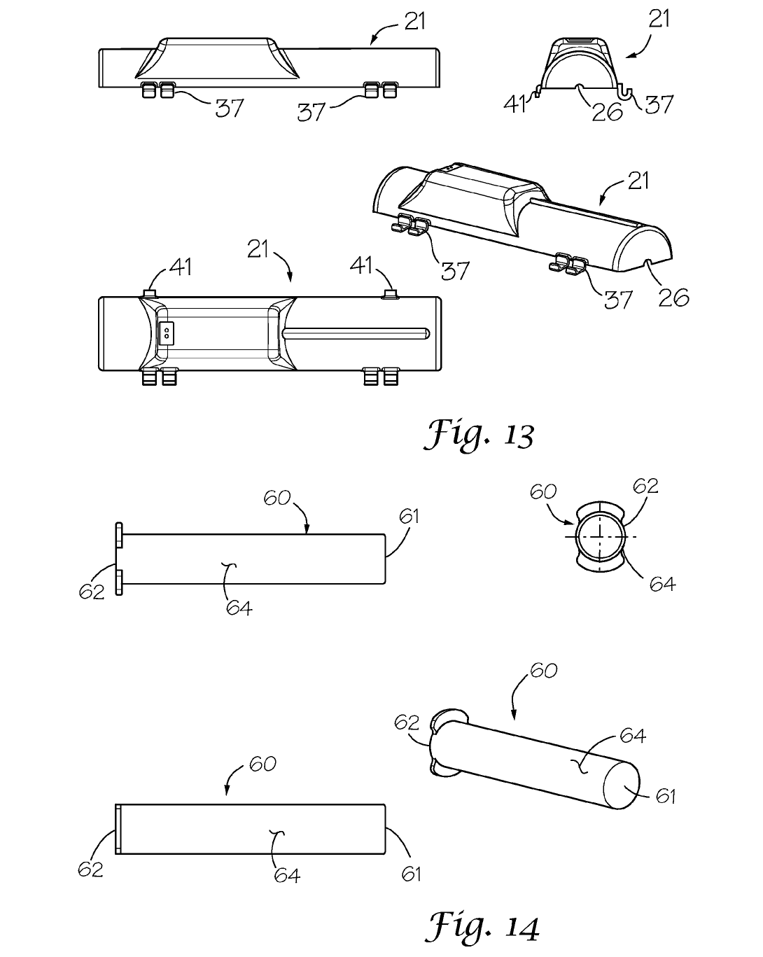

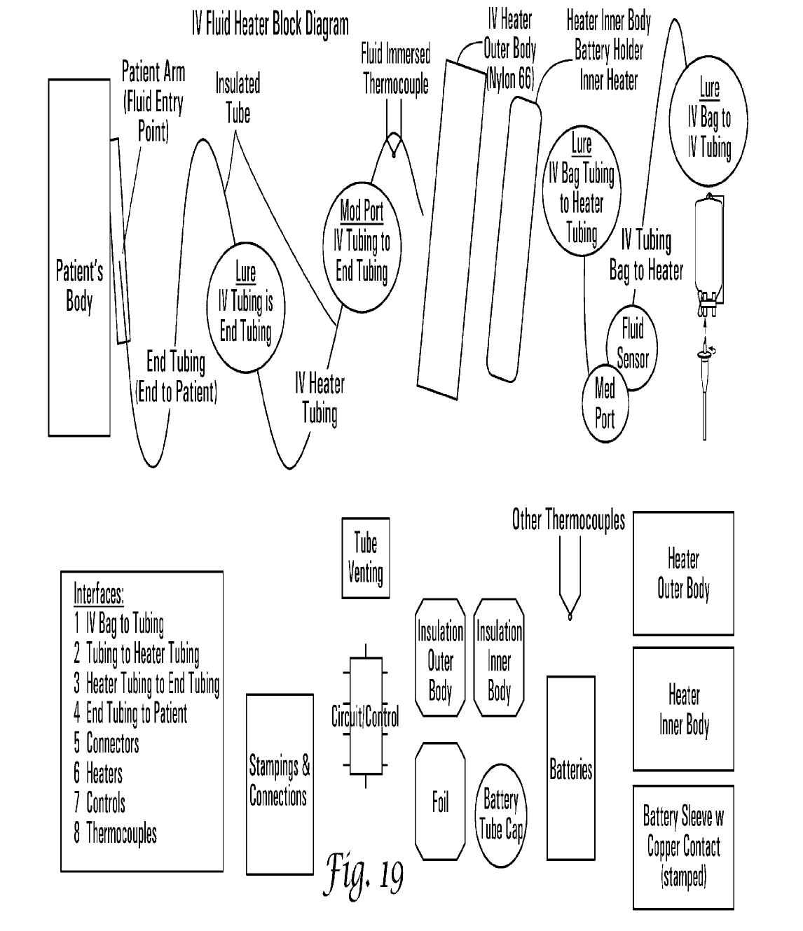

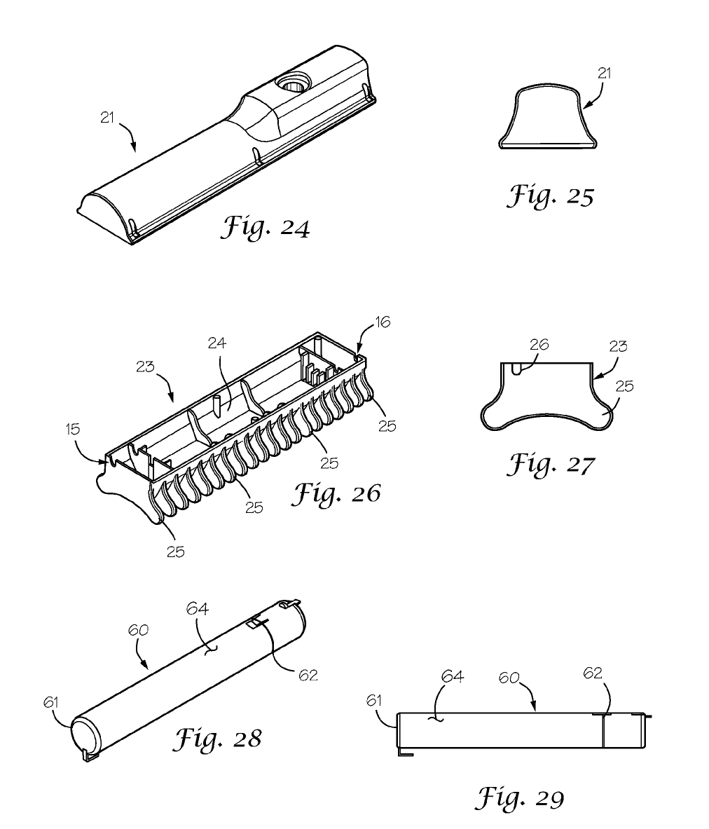

FIGS. 1-30 depict various embodiments of a heat transfer device (i.e., warmer, heater, fluid heater, intravenous fluid heater, etc.) 10 according to the present disclosure having particular utility in the heating of a fluid (in particular a liquid) flowing in an intravenous tube 90. The heater 10 is designed to generate heat and transfer the heat to a fluid (such as a saline solution, medicament, blood other fluid delivered via an intravenous line to a patient) flowing through an intravenous fluid tube 90. The heater 10 is particularly designed to optionally be wearable by a patient that will be receiving the intravenous fluid being warmed by the heater 10. The fluid heater 10 has utility in extreme weather conditions as occur in relatively extremely cold and remote locations including, in particular, relatively high altitude and/or mountainous regions. Further, the fluid heater 10 has particular utility for medical personnel (including military medics) for treating injured individuals (including soldiers and civilians) in a variety of settings (such as in a remote and cold mountain location where a soldier may be injured in action).

Referring now to the drawings, and referring in particular to FIGS. 1 through 9, there is disclosed a fluid heater 10 including a housing 12 (preferably made from a hard plastic material, such as polypropylene (PPO) in the shape of a generally round or cylindrical shape. The heater 10 is designed to be wearable by a patient and is highly portable and efficient. In one embodiment, the heater 10 has a generally cylindrical external shape as best shown in FIGS. 4 and 10 but the heater 10 may have any appropriate alternative, external shape and may have additional features incorporated therein as more fully described herein. The housing 12 may be made of any known or appropriate material such as a thermoplastic, metal, alloy or other material.

In the one exemplary embodiment, the housing 12 includes a first or top housing member 21 and second or bottom housing member 23. In the one exemplary embodiment, the first and second housing members 21 and 23, respectively, each have a generally elongated, half-round (or half-cylinder) shape so that when coupled together they generally form a cylinder shape. It should be understood that the first and second housing members 21 and 23, respectively, need not have the same, symmetrical or similar shapes and that they may be designed to have other non-cylindrical shapes.

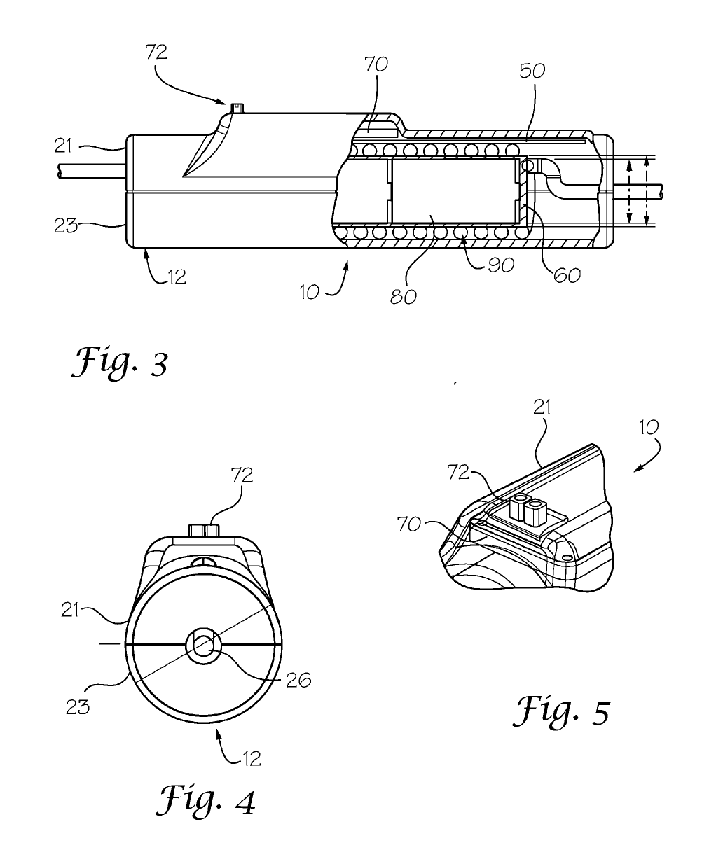

In one exemplary embodiment, the first and second housing members 21 and 23, respectively, are coupled or joined on at least one side by a hinge 36 or pair of hinges 36, but may alternatively be coupled together using any known or appropriate coupler, fastener or joint. In one embodiment, hinges 36 includes a first or upper hinge portion 37 and a mating, second or lower hinge portion 38. The hinge portions 37, 38 are pivotably coupled to each other via conventional devices (e.g., bayonet, tongue and groove, rod, bolt, screw, or other similar means, not shown). The first and second housing members are coupled or joined together on another side thereof by a pair of latches 41 (FIG. 2) but may alternatively be coupled together using any known or appropriate coupling, fastener or joint that will work in conjunction with the hinges 37 and 38 to effectively close the housing 12 and can also be unlatched to open the housing 12. In FIGS. 1 and 2, the latches 41 are each shown as a latch including a barbed (or pronged) extension member received in a receptacle where the extension member is inserted in a passage in the receptacle and the barb engages or catches a surface of the receptacle to couple the first and second housing members 21 and 23 together. The latch 41 is designed to have an engagement (or alternatively an interference fit) between the first and second housing members 21 and 23 such that there is a biasing force between the extension member and receptacle to maintain the latch 41 in the latch position. Other types of latches or couplings may be used to maintain the first and second housing members 21 and 23 in a closed position. Further the first and second housing members 21 and 23 are preferably sealed using a grommet or other type of seal (not shown). As best shown in FIGS. 3-9, in one alternate embodiment, it is possible to have the housing 12 have no hinge and/or no latch. Instead, the housing 12 may be sealed shut using any known or appropriate means such as adhesives, chemical bonding, welding, grommets, gaskets and the like.

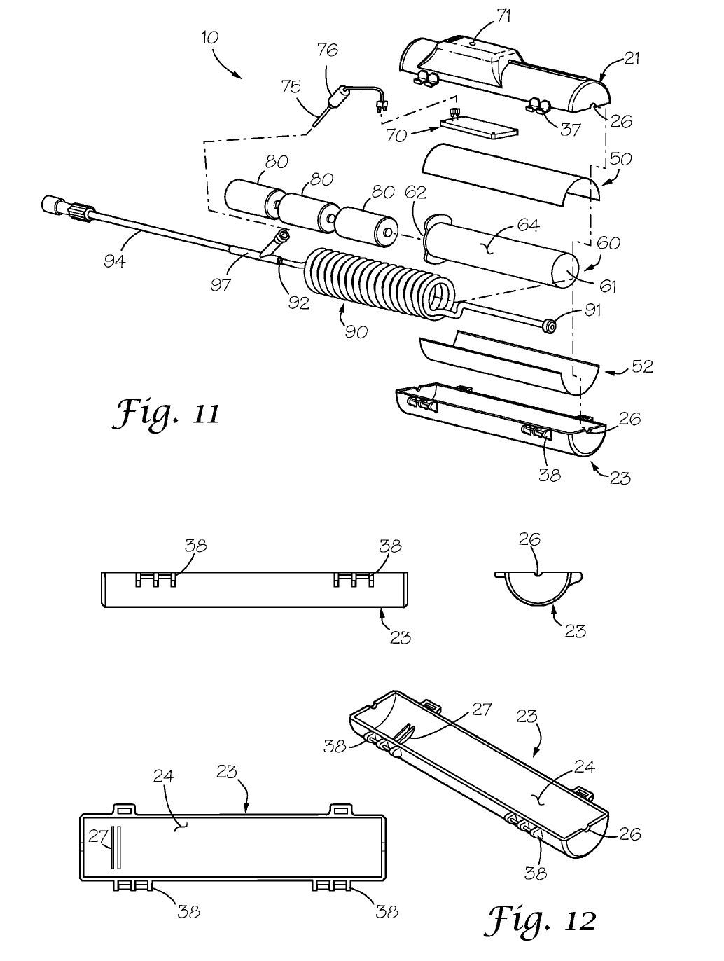

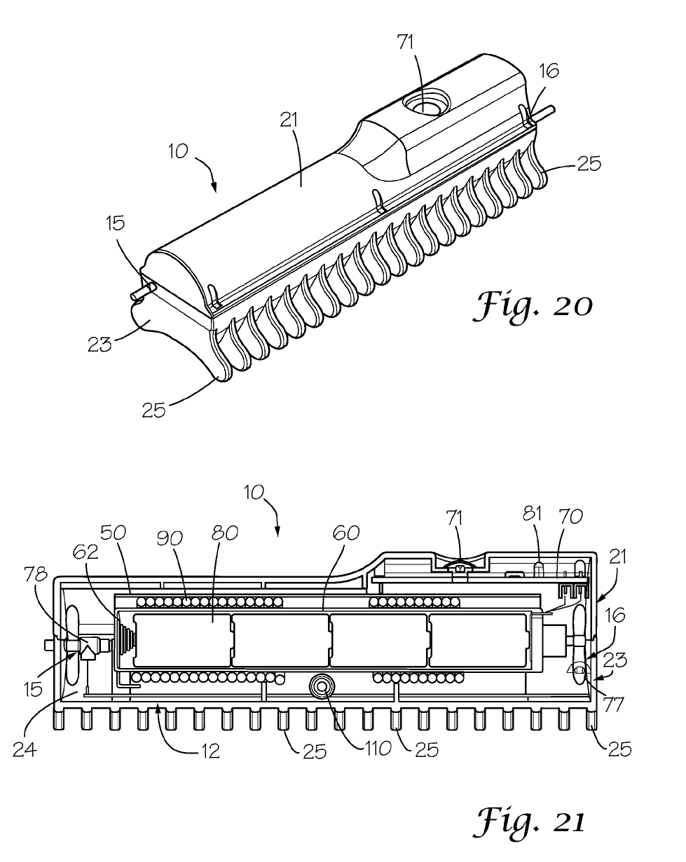

In one exemplary embodiment, the first and second housing members 21 and 23, respectively, each have an interior surface 24 which generally defines a space within the housing 12. The interior surfaces 24 of the first and second housing members 21 and 23 is a generally continuous, smooth surface but may include any number of features. The interior surfaces 24 of the first and second housing members 21 and 23 generally define a cylindrical area when the housing 12 is closed but may alternatively define any other shape as necessary or appropriate. Referring to FIG. 12, in one exemplary embodiment, the interior surfaces 24 of the first and second housing members 21 and 23 each include a plurality of rib or extension members 27 projecting generally from the surface 24 over a given extent of each of the first and second housing members 21 and 23, respectively, for supporting components within housing 12. Referring to FIGS. 20 and 21, in one embodiment, second housing member 23 includes a plurality of legs 25 extending outwardly to support heater 10 and prevent movement when placed on the ground.

In one exemplary embodiment, the first and second housing members 21 and 23, of the housing 12, respectively, each have a first end and a second end, and each end has an access means, opening, hole, or passage 26 therein for routing the intravenous tube 90 or portion of the intravenous tube 90 through the housing 12. Referring to FIG. 4, in one exemplary embodiment, the passage 26 is located centrally on one end of the housing 12 and is therefore sectioned approximately in half and has a portion located on each of the first and second housing members 21 and 23, respectively. On the other end of the housing 12, the passage is located offset from the center of the end but still along the parting line between the first and second housing members 21 and 23. In each instance, the passage 26 is preferably designed to have a shape and size that is matched to the intravenous tube 90 outer diameter such that the passage 26 will substantially create a seal when the first and second housing members 21 and 23 are closed. Any alternative design for the passage 26 is possible provided it is useful to allow the intravenous tube to pass through the housing 12 while functioning with the heater 10 to properly heat the intravenous fluid.

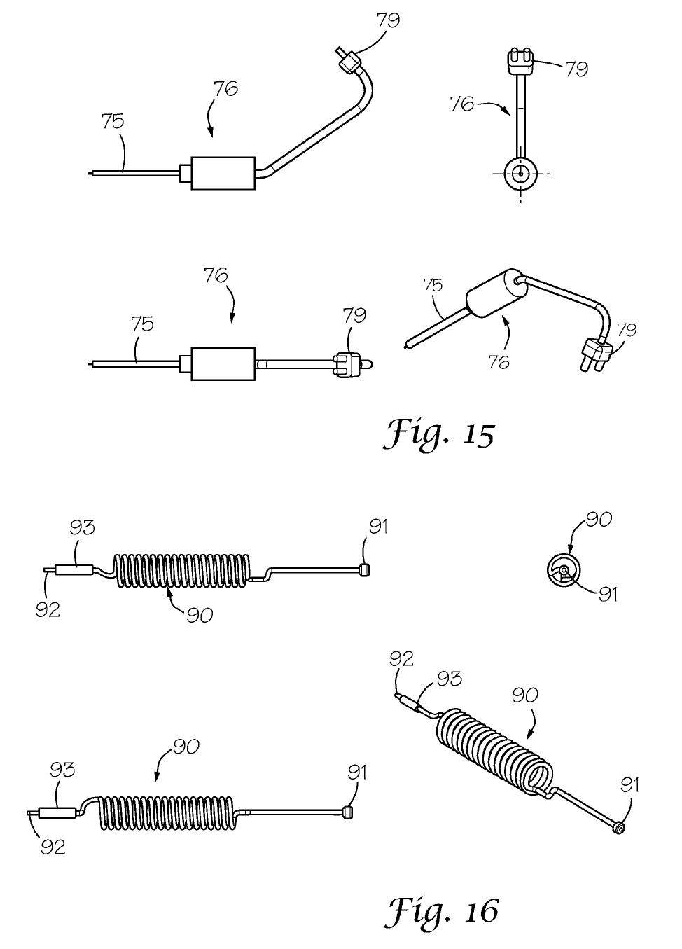

In one exemplary embodiment, the intravenous tube 90 is preferably a standard, sterilized plastic material as is typically used in medical intravenous tube applications. Referring to FIG. 15, in one exemplary embodiment, the intravenous tube 90 or a portion thereof within the housing 12, may be made of a metal material such as an aluminum tube wrapped around the inner housing 60. In particular, as explained below, the metal-based intravenous tube 90 is preferably aligned with the heater elements of the heater 10 for improving the heat transfer from the heater elements to the fluid flowing in the intravenous tube 90. Alternatively, the intravenous tube 90 may be made from a stainless steel or other metal or alloy material appropriate for the given purpose. The intravenous tube 90 within the housing 12 includes an inlet connector end 91 and an outlet connector end 92.

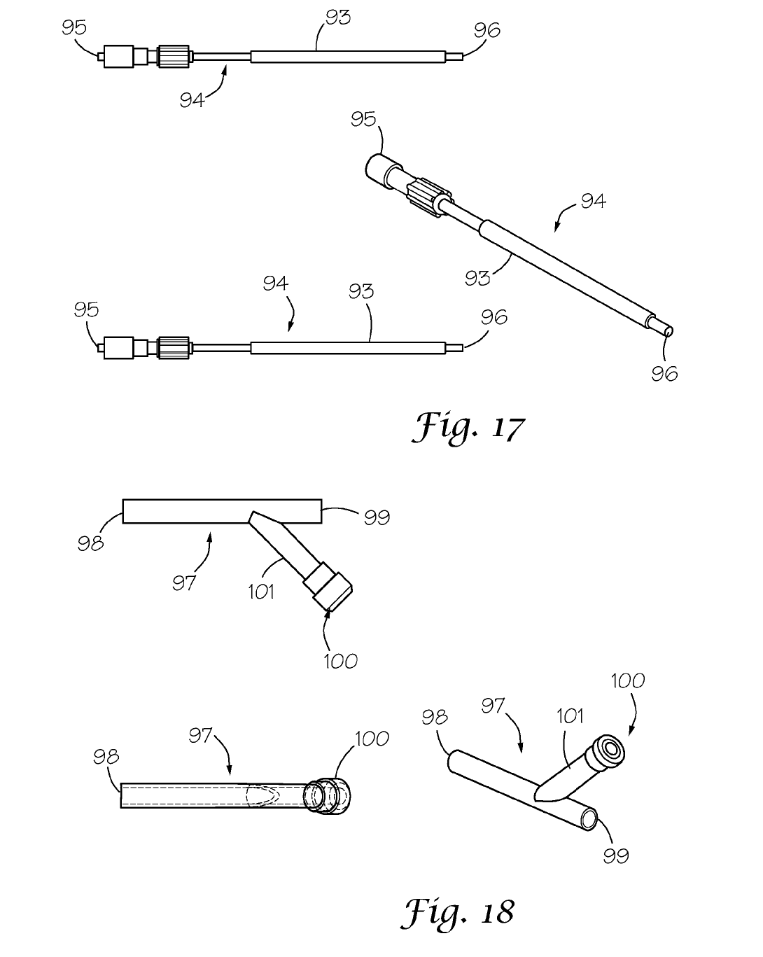

The heater 10 is intended for use in austere weather environments and therefore in one embodiment, with reference to FIGS. 16 and 17, the intravenous line 90 may include an insulated intravenous tube portion 94 exiting from the heater 10 and for connecting the intravenous line 90 to a patient. The insulated intravenous tube portion 94 exiting the heater 10 may be insulated to limit and/or prevent heat from exiting the intravenous fluid therein. Accordingly, an insulator 93 is applied around at least a portion of the intravenous line 90 that exits heater 10 to define insulated intravenous tube portion 94. Insulator 93 may be made of any known or appropriate thermally insulative material useful in preventing or limiting heat from being transferred from the heated intravenous fluid exiting the heater 10. Further, given the relatively thermally insulative nature of the known and standard materials used in an intravenous line 90, it is possible to form the insulator 93 as a thickened wall portion of the intravenous line 94. Referring to FIGS. 11 and 16-18, in one embodiment, distal end 96 of insulated intravenous tube portion 94 connects to second connector end 98 of y-connector 97, and outlet connector end 92 of intravenous line 90 exiting housing 12 at outlet port 16 connects to first connector end 99 of y-connector 97. Second distal end 95 of insulated intravenous tube portion 94 is preferably adapted with an appropriate connector to facilitate connecting to the patient.

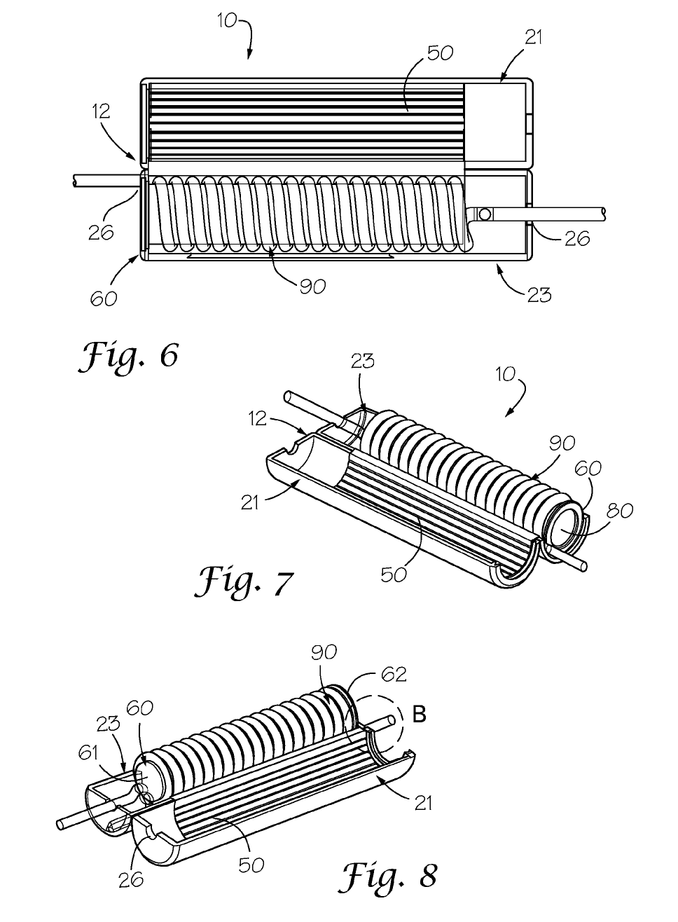

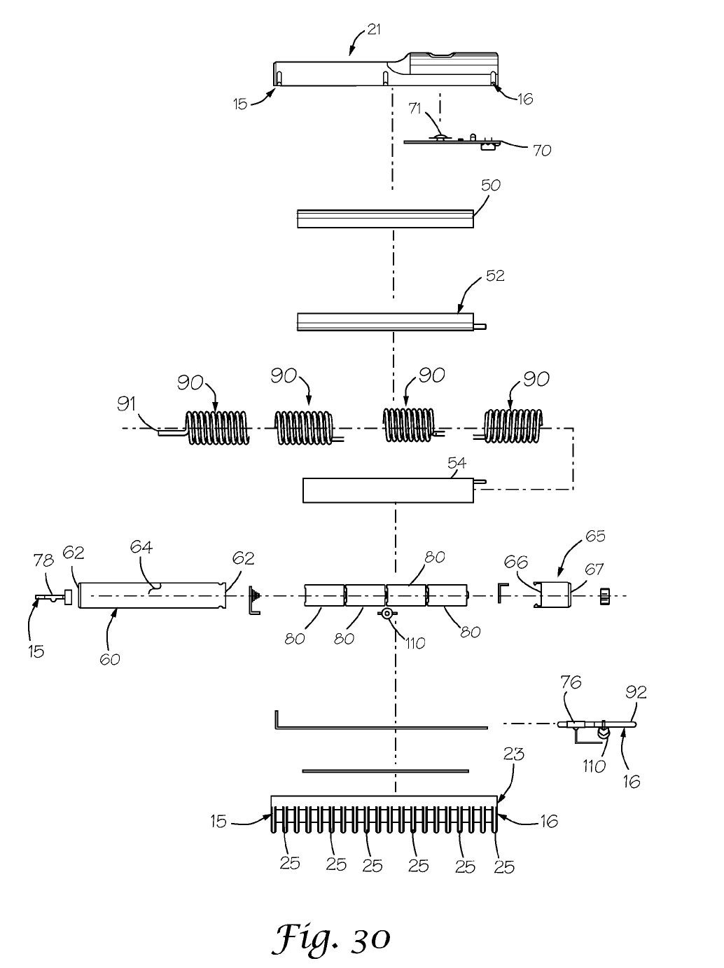

In the exemplary embodiments, the heater 10 includes a heater apparatus or assembly including a first (or at least one) heating element 50 which is preferably located in the first or upper housing member 21 as best shown in FIGS. 6-9 and 21-22. In one exemplary alternate embodiment, the heater 10 further includes a second or lower heating element 52 which is preferably located in the second or lower housing member 23, as best shown in FIGS. 11 and 30. The heaters 50 and 52 are preferably isolated from the first and second housing members 21, 23 using a relatively thin insulating layer. The insulating layer may be made from any known or appropriate material useful to help impede the transfer of heat from the heating element 50, 52 to the housing members 21, 23, respectively. In one particular embodiment, the insulating layer is primarily made from any known or appropriate insulating materials such as a neoprene rubber or urethane-based material and may be coupled to the inner side of the housing members 21, 23 using any known or appropriate adhesive material such as an acrylic-based adhesive material that may alternatively designed as a pressure sensitive layer for adhering the heater elements 50, 52 to the housing members 21, 23. In one exemplary embodiment, the adhesive may even be a peel-n-stick layer for improving the manufacturability of the heater 10. Alternatively, the adhesive material may be a mechanical connection or a chemical bonding agent or the like or any other known or appropriate way of coupling the heater elements 50,52 to the housing members 21, 23.

In one exemplary embodiment as best shown in FIG. 30, there is a heater 10 disclosed wherein the heating apparatus includes three heater elements 50, 52 and 54. The first and second heater elements 50 and 52 are similar to the exemplary embodiment of FIGS. 1 through 19, wherein they are located on the interior surfaces 24 of the first and second housing members 21 and 23. The third heater element 54 is located on the exterior surface 64 of the battery housing 60 (center cylinder).

All three heater elements 50, 52, 54 may be turned on together or individually depending upon the flow rate of the intravenous fluid read by the intravenous fluid flow sensor 78 and the readings from the first and second temperature sensors 76, 77 used to determine the amount of heat required to heat the intravenous fluid to the desired output temperature of the heater 10.

The heater elements 50, 52 and 54 are preferably a thin-film type heater member capable of converting energy in the form of electrical current to generate heat in the manner set forth and described in International Publication Number W02008/122043, in the name of Saunders et al., entitled Heater Device, and published Oct. 9, 2008; and claiming priority to U.S. Provisional Patent Application No. 60/909,590, in the name of Saunders et al. and filed Apr. 2, 2007, the entire contents of which are incorporated herein by reference.

The heater elements 50, 52 and 54 include a substrate composed of an electrically insulative material and a resistive material layer having a sheet resistance of 10 ohm/sq to 20,000 ohm/sq that is defined on at least a portion of at least one surface of the substrate. The heater elements 50, 52 and 54 include a bus structure in communication with the resistive material layer that has at least two elements and is configured to introduce current into the resistive layer with the current traveling in at least one current path in the resistive layer. In one exemplary embodiment, the heater 10 preferably includes a flex circuit coupled to the heater elements 50, 52 and 54 for electrically connecting the heater elements 50, 52 and 54 with the energy supply and the control circuit. Alternatively, any other known or appropriate electrical conducting members may be used in completing the electrical circuit among the controller, heater elements 50, 52 and 54 and the energy source.

In one exemplary embodiment, the heater material of the heater element 54 (and alternatively of the heater elements 50, 52) may be directly applied to the exterior surface 64 of the inner housing member 60 using a spray deposition technique of a carbon-based resistive material for generating heat when an electrical current flows there through. Appropriate electrical connections may then also be applied for electrically coupling the heater 54 to the controller 70 and the energy source 80.

In one exemplary embodiment, the heater 10 includes an inner housing 60 (battery housing) located internal of the first and second housing members 21, 23, respectively. In one exemplary embodiment as shown in FIGS. 11, 14, 21, and 28 through 30 the inner housing 60 preferably is completely contained within the housing 12 once the housing 12 is closed but may be coupled with the housing 12 in other ways such that it is not completely contained therein but is still sealed. It is preferable to provide a complete seal of the battery housing 60 and its contents from external elements (such as the weather, etc.). The battery housing 60 may be made of any known or appropriate material such as a thermoplastic, or other material having a relatively higher insulating characteristic and is preferably not made from a metal, alloy or aluminum tubing which provides improved heat transfer characteristics.

The battery housing 60 is preferably formed as an elongate, hollow member having a substantially round cross section, having a first (or closed) end 61 and a second (or open) end 62 in the exemplary embodiments of FIGS. 11, 14, and 21. In the exemplary embodiment of FIGS. 21 and 28 through 30, the battery housing 60 includes a cap or closure member 65 designed to be coupled to the second end 62 of the inner housing 60 as best shown in FIGS. 21 and 30. Closure member 65 includes an open end 66 receiving second end 62 of inner housing 60, and a closed end 67 that encloses batteries 80 in housing 60 when closure member 65 is attached to second end 62.

The battery housing 60, in the exemplary embodiments of FIGS. 11, 14, 21, and 28 through 30, is substantially cylindrically-shaped and is located substantially concentrically with the outer housing 12 to form a ring-shaped passage 63 between the exterior surface 64 of the inner housing 60 and the inner surface of the exterior housing 12. The battery housing 60 is designed to hold one or more batteries for providing power to the heater 10 as described below. The battery housing 60 may be sized to optimally fit any known or appropriate type of energy source or battery 80 but is preferably sized to receive a particular known standard size battery and, more particularly, is preferably sized to receive three (FIGS. 1 through 18) or four (FIGS. 20 through 30) size A123 batteries 80 for supplying electrical current to the heater device.

The heater 10 includes the controller 70 which is electrically coupled to the various components of the heater 10 using an electrical circuit. The controller 70 is for controlling or adjusting the amount of energy (or power such as electrical current) provided to the heater elements 50, 52 and 54, as provided, to control the amount of heat generated and thereby, the amount of heat transferred to the intravenous tube 90 and the intravenous fluid flowing therein. The controller 70 includes inputs from the first and second temperature sensors 76 and 77. The controller 70 includes an appropriate algorithm for comparing the inputs from the first and second temperature sensors 76, 77 and for determining the amount of energy to be supplied by the batteries 80 to the heater apparatus 50 for generating heat to be applied to the intravenous tube 90 to raise the temperature of the intravenous fluid flowing through the heater 10.

In one exemplary embodiment, the heater 10 includes at least one vent 110 located within the housing 12 and coupled to the intravenous tube 90. The vent 110 is coupled within the intravenous line 90 to vent gas from the intravenous fluid prior to the intravenous fluid exiting the heater 10. The vent 110 can also vent gas from the intravenous fluid to balance the pressure within the heater 10 with the ambient pressure outside of the heater 10.

The heater 10 is operated by an on-off (or heater control) switch 71 coupled to the controller 70 and using any known or appropriate design. The switch 71 functions to selectively supply power to the controller 70 from the energy source 80 which also functions to supply power from the energy source 80 to the heater elements 50, 52 and 54 in normal operating conditions. A microprocessor may be included in the controller 70 of the heater 10 to facilitate a pulse width modulation of the output voltage or to otherwise regulate the current of the battery cells 80 as applied to the heater elements 50, 52 and 54 in any known or appropriate manner. The controller 70 may selectively interrupt the switch 71 or otherwise alter the supply of energy to the heater elements 50, 52 and 54 based on a range of conditions stemming from personal safety and circuit operation considerations.

In one exemplary embodiment, a first temperature sensor 76 is included at an outlet 16 of the heater 10 and is monitored by the controller 70 to detect if the intravenous fluid at the heater outlet 16 is exceeding a predetermined limit (i.e., approximately 43° C.), wherein the heater 10 will cease the supply of energy (i.e., electricity) to the heater elements 50, 52 and 54 to prevent an overheating condition of the intravenous fluid within the intravenous tube 90. As best shown in FIGS. 10 and 11, in the illustrated embodiment, first temperature sensor 76 is carried by a y-connector 97 disposed along intravenous line 90 at outlet port 16. Referring to FIG. 18, y-connector 97 includes a first connector end 99 receiving intravenous line 90 from outlet port 16 of the heater 10, and a second connector end 98 connecting to an additional intravenous line 90 for delivery of the heated intravenous fluid. Secondary connector port 100 is provided on the y-arm 101 for receiving first temperature sensor 76. With further reference to FIG. 15, first temperature sensor 76 includes a probe 75 which extends down y-arm 101 to measure the intravenous fluid temperature. First temperature sensor 76 further includes a connector 79 for engaging sensor plug 72 (best shown in FIG. 5), which is electrically coupled to the controller 70. In an alternative embodiment, plug 72, or an additional such connector port, can be operable to connect to an external power supply for powering heater 10. Additionally, a second temperature sensor 77 may be included in the heater 10 at any appropriate location such as at inlet 15 of the heater 10. The second temperature sensor 77 is in fluid communication with intravenous fluid passing into heater 10 along intravenous line 90. Further, second temperature sensor 77 is also electrically coupled with and monitored by the controller 70 for use in controlling the heater elements 50, 52 and 54 for affecting the total heat transfer to the intravenous fluid flowing in the intravenous line 90.

In one exemplary embodiment, a fluid present sensor 78 is provided in the heater 10 for detecting whether the intravenous fluid is flowing within the intravenous line 90. In the disclosed embodiments, the fluid present sensor 78 is disclosed as being located on the inlet 15 of the heater 10 but may alternatively be located within the housing 12 of the heater 10 at any known or appropriate location. Further, the fluid present sensor 78 may be located at the outlet 16 of the heater 10. The fluid present sensor 78 may be of any known or appropriate type such as an optics-based or magnetic-based sensor for determine that there is at least a threshold flow rate of the intravenous fluid. In one exemplary embodiment, the fluid present sensor 78 is coupled with the controller 70 for providing an input to the controller indicating whether the intravenous fluid is flowing above a predetermined value. The heater 10 and the controller 70 are also responsive to the inputs from a flow sensor for measuring the flow of the intravenous fluid within intravenous tube 90 and a pressure sensor for measuring the pressure within the heater 10. The controller 70 receives inputs from the pressure and flow sensors for determining the control of the heater 10 while managing the temperature of the intravenous fluid exiting the heater 10.

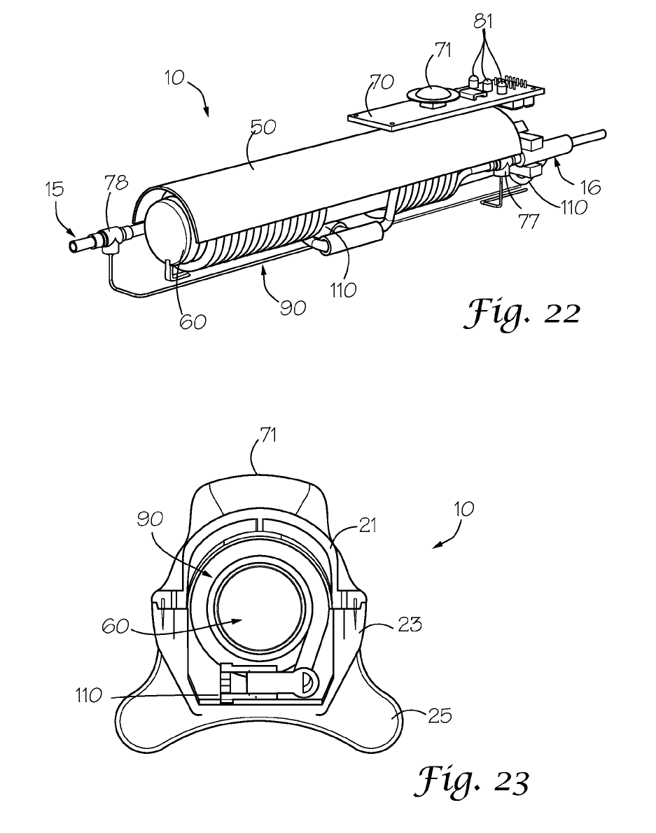

In one exemplary embodiment, the controller 70 is designed to monitor the state of the energy source or batteries 80, such as battery life remaining, and include such information in determining the energy load to be applied to the heater elements 50, 52 and 54 such as by varying the pulse-width modulation of the current being drawn from the batteries 80. In one exemplary embodiment, the heater device 10 may be provided with an audible alarm for audibly signifying a nearly or fully discharged state and/or a hazardous state. Referring to FIG. 22, multicolor LEDs 81 can be included to show, for example, a change from red to green to indicate the state of charge.

The controller 70 may further include additional sensors (or inputs from additional sensors) including: a voltage sensor, a current sensor, and a strain or pressure sensor based on a given application by making simple changes in the heater 10 and the controller 70 appreciated by a person having an ordinary skill in the art.

It should be appreciated that with the design of the heater 10, particularly where the housing 12 is designed to be relatively easily opened by an operator, it is possible to open the housing 12 and remove the battery housing 60 along with the batteries 80 along with the intravenous tube 90 can be relatively easily removed from the housing 12 and then disposed or sent for recycling. Next, a new battery housing 60 along with new batteries 80 and a new intravenous tube 90 can be relatively easily reinserted into the housing 12 and the housing 12 then closed and the heater 10 used again in the field. In one exemplary embodiment, the heater 10 and its components are designed to be entirely disposed or recycled of after a single use.

The intravenous fluid warmer or heater 10 of the disclosure is a complete, functioning device that is designed to contain and function using standard, off-the-shelf, battery cells, while providing appropriate thermal control and monitoring circuitry for reliable and safe operation without a need for auxiliary or additional equipment. In one exemplary embodiment, the intravenous fluid warmer assembly may have a replaceable heater cartridge inside intravenous fluid warmer or heater 10 is designed to as a replaceable heater cartridge wherein the batteries and the battery housing 60 may be removed from the housing 12. Further the intravenous fluid line components in contact with the intravenous fluid are also of a single-use design considering a convenient use or medically hazardous conditions.

While a preferred embodiment of the invention has been described using specific terms, such description is for illustrative purposes only, and it is to be understood that changes and variations may be made without departing from the spirit or scope of the following claims.

What is claimed is:

1. A heat transfer apparatus for use in heating an intravenous fluid to be administered to a body, the heat transfer apparatus comprising:
a housing configured for having an intravenous line enter at a first point and exit at a second point;
an energy source for supplying energy to the heat transfer apparatus, the energy source being located within the housing; and
a heat transfer element for producing heat to be transferred with the intravenous line to change the temperature of the intravenous fluid flowing through the intravenous line.

2. The heat transfer apparatus of claim 1 wherein the heat transfer apparatus is located proximal the housing and the energy source is located more toward the center of the housing than the heat transfer apparatus and the intravenous line is located between the heat transfer apparatus and the energy source.

3. The heat transfer apparatus of claim 1 wherein the heat transfer element and the intravenous line are pressed together to increase the effective heat transfer with the fluid in the intravenous line.

4. The heat transfer apparatus of claim 1 further comprising an inner housing member located within the housing and the energy source is located within the inner housing and heat transfer element and the intravenous line are pressed together by the housing and the inner housing.

5. The heat transfer apparatus of claim 4 wherein the housing and the inner housing each have a substantially round cross section located concentrically to define a space there between and the heat transfer element and the intravenous line are located within the space.

6. The heat transfer apparatus of claim 5 wherein said space is substantially cylindrical shaped.

7. The heat transfer apparatus of claim 1 wherein the intravenous line is coil wrapped around the inner housing.

8. The heat transfer apparatus of claim 7 wherein the coils of the coil wrapped intravenous line are spaced apart to increase the heat transfer to a fluid flowing in the intravenous line.

9. The heat transfer apparatus of claim 1 further comprising a temperature sensor for monitoring the temperature of a fluid exiting the heat transfer apparatus and a display coupled to the heat transfer apparatus, the display for indicating the monitored status of the temperature sensor.

10. The heat transfer apparatus of claim 1 further comprising a temperature sensor for monitoring the temperature of a fluid exiting the heat transfer apparatus for controlling the energy from the energy source being supplied the heat transfer element for maintaining the temperature of the fluid within a defined temperature range.

11. An apparatus for changing the temperature of a fluid to be flowing in an intravenous line to be administered intravenously to a body, the apparatus comprising:
a housing configured for having the intravenous line enter and exit the housing;
a heat transfer element for transferring heat with the intravenous line to change the temperature of the fluid in the intravenous line;
an energy source for supplying energy to the heating element to produce heat, the energy source being located within the housing;
a temperature sensor for monitoring the temperature of the fluid exiting the apparatus;
a controller coupled to the housing for controlling the supply of energy from the energy source to the heating element in response to the temperature sensor; and
a display coupled to the apparatus, the display for indicating the status of the apparatus.

12. The apparatus for changing the temperature of a fluid to be flowing in an intravenous line of claim 11 wherein the housing includes a portion that is at least partially translucent and the display is arranged in the housing to be viewable through the translucent portion of the housing.

13. The apparatus for changing the temperature of a fluid to be flowing in an intravenous line of claim 11 wherein the display indicates a status of the energy source and provides a temperature indicator of the intravenous fluid.

14. The apparatus for changing the temperature of a fluid to be flowing in an intravenous line of claim 11 further comprising a vent system located within the housing, the vent system being coupled with the intravenous line to vent gas from the intravenous fluid prior to exiting the apparatus.

15. The apparatus for changing the temperature of a fluid to be flowing in an intravenous line of claim 14 wherein the vent system vents gas from the intravenous fluid to balance the pressure within the apparatus with the ambient pressure outside of the apparatus.

16. An apparatus for heating an intravenous fluid in an intravenous line, the apparatus comprising:
a housing having an opening for receiving a length of the intravenous line;
a heat transfer element for producing heat to be transferred to the intravenous line to change the temperature of the intravenous fluid flowing through the intravenous line; and
a battery cell located within the housing for providing electrical power to the heat transfer element; and
wherein the intravenous line is located between the battery cell and the heat transfer element.

17. The apparatus of claim 16 further comprising an inner housing having the battery cell located therein and wherein the housing is an outer housing and the heat transfer element is located proximal the outer housing and the intravenous line is located between the heat transfer element and the inner housing.

18. The apparatus of claim 17 wherein the heat transfer element and the intravenous line are pressed together to increase the effective heat transfer to the fluid in the intravenous line.

19. The apparatus of claim 18 wherein the outer housing and the inner housing each have a substantially round cross section located concentrically to define a generally cylindrical shape space and the heat transfer element and the intravenous line are located within the space.

20. The apparatus of claim 19 wherein the intravenous line is coil wrapped around the inner housing.

21. The apparatus of claim 20 further comprising an insulator located on the outer surface of the inner housing and an insulator located on the inner surface of the outer housing.

22. The apparatus of claim 16 further comprising a temperature sensor for monitoring the presence of a fluid in the intravenous line and controlling the supply of electrical power from the battery cell to the heat transfer element.

23. The apparatus of claim 16 further comprising a temperature sensor for maintaining the temperature of the fluid exiting the apparatus within a defined temperature range.

24. A portable apparatus for providing energy to heat an intravenous fluid, the portable apparatus comprising:
a housing having an opening to receive a length of a flexible intravenous tube for connecting a source of intravenous fluid to a body;
an energy source comprising a battery cell;
a heating element for converting energy from the energy source to heat for heating the intravenous fluid flowing in the intravenous tube, and
wherein the intravenous tube is at least partially wrapped around the battery cell.

25. A apparatus for providing heat to change the temperature of an intravenous fluid, the apparatus comprising:
a housing having an opening to receive a length of a flexible intravenous tube for connecting a source of intravenous fluid to an intravenous catheter;
a heat transfer element for transferring heat with the intravenous tube the intravenous fluid flowing in the intravenous tube;
an energy source for supplying energy to the heat transfer element;
an energy source receptacle having at least a portion of the energy source located therein, the energy source receptacle being located within the housing; and,
wherein the intravenous tube has at least a portion extending between the heat transfer element and the energy source receptacle.

26. The apparatus of claim 25 wherein the housing includes a latch that can be opened to open the housing and the energy source and the intravenous tube can be removed from the housing and replaced with a new energy source and intravenous tube.

27. The apparatus of claim 26 wherein the intravenous tube has at least a portion of the intravenous tube exiting the apparatus and the apparatus further comprises an insulator covering the portion of the intravenous tube exiting the apparatus for limiting heat transfer from the heated intravenous fluid exiting the apparatus.
